(19) United States
(12) Patent Application Publication
(10) Pub. No.: US 2023/0233130 A1
(43) Pub. Date: Jul. 27, 2023

(54) Heart Rate Detection Method, Device, and Program

(71) Applicant: Nippon Telegraph and Telephone Corporation, Tokyo (JP)

(72) Inventors: Nobuaki Matsuura, Tokyo (JP); Kei Kuwabara, Tokyo (JP); Yuichi Higuchi, Tokyo (JP); Takako Ishihara, Tokyo (JP)

(21) Appl. No.: 18/007,939

(22) PCT Filed: Jun. 23, 2020
(86) PCT No.: PCT/JP2020/024528

§ 371 Date: Dec. 2, 2022

(51) Int. Cl. A61B 5/352; A61B 5/00

(52) U.S. Cl. CPC A61B 5/352; A61B 5/742

(57) ABSTRACT

According to the present disclosure, a heart rate detection method includes measuring an ECG waveform, calculating an R-R interval from the ECG waveform, calculating an instantaneous heart rate from the R-R interval, calculating an index based on the instantaneous heart rate, and determining whether the heart rate obtained from the R-R interval is to be displayed, by comparing the index to a reference value.

### CROSS-REFERENCE TO RELATED APPLICATIONS

This application is a national phase entry of PCT Application No. PCT/JP2020/024528, filed on Jun. 23, 2020, which application is hereby incorporated herein by reference.

### TECHNICAL FIELD

The present disclosure relates to a heart rate detection method, apparatus, and program for presenting an accurate heart rate.

### BACKGROUND

The heart rate or the fluctuation of the heart rate is biological information obtained from an electrocardiogram (ECG), and is utilized for evaluation of autonomic nerve function, an index of exercise intensity, and the like in a daily life and a resting state.

In particular, measuring the heart rate over a relatively long time in a daily life, or the like by using a wearable ECG waveform acquisition/heartbeat measurement device is useful for physical condition management of a user and the like.

PTL1 discloses a method capable of appropriately detecting a heartbeat in a case where noise is superimposed on an ECG waveform, such as measurement by a wearable device.

Unfortunately, the heartbeat may be erroneously detected in this method in a case where the noise of the ECG waveform due to a body motion is large. For example, in a case where an R wave to be detected is missed, a wave that is not the R wave may be detected as the R wave.

To avoid such an error in heartbeat detection, a wearable heartbeat measurement device may adopt a method of calculating and presenting an average heart rate in which fluctuation of an instantaneous heart rate caused by an error in heartbeat detection is suppressed by moving-averaging the heart rate over a predetermined time or using an IIR filter, and the like (PTL 2).

### Citation List

### Patent Literature

PTL 1: JP 6527286B
PTL 2: JP 6645926B.

### SUMMARY

### Technical Problem

In the heartbeat detection method disclosed in PTL2, a value such as an average heart rate is calculated for any ECG waveform data. Thus, even in a situation where the heartbeat is erroneously detected and thus is not measured as an accurate value, an abnormal numerical value may be presented to the user as an accurate value.

### Means for Solving the Problem

In order to solve the problem as described above, according to the present disclosure, there is provided a heart rate detection method including measuring an ECG waveform, calculating an R-R interval from the ECG waveform, calculating an instantaneous heart rate from the R-R interval, calculating an index based on the instantaneous heart rate, and determining whether the heart rate obtained from the R-R interval is to be displayed, by comparing the index to a reference value.

In addition, according to the present disclosure, a heart rate detection apparatus includes a measurement unit configured to measure an ECG waveform, a calculation unit configured to calculate an R-R interval from the ECG waveform, calculate an instantaneous heart rate from the R-R interval, and calculate an index based on the instantaneous heart rate, and a determination unit configured to determine whether the heart rate obtained from the R-R interval is to be displayed, by comparing the index to a reference value.

In addition, according to the present disclosure, there is provided a heart rate detection program causing a heart rate detection apparatus to operate, the heart rate detection program causing the heart rate detection apparatus to perform measuring an ECG waveform, calculating an R-R interval from the ECG waveform, calculating an instantaneous heart rate from the R-R interval, calculating an index based on the instantaneous heart rate, and determining whether the heart rate obtained from the R-R interval is to be displayed, by comparing the index to a reference value.

### Effects of Embodiments of the Invention

According to the present disclosure, it is possible to provide a heart rate detection method, apparatus, and program for presenting an accurate heart rate.

### BRIEF DESCRIPTION OF THE DRAWINGS

FIG. 1 is a diagram illustrating an ECG waveform and an R-R interval for describing a heart rate detection method according to a first embodiment of the present disclosure.

FIG. 2 is a diagram illustrating an instantaneous heart rate and an average heart rate for describing the heart rate detection method according to the first embodiment of the present disclosure.

FIG. 3 is a diagram illustrating a temporal change of an index in the heart rate detection method according to the first embodiment of the present disclosure.

FIG. 4 is a block diagram illustrating a heart rate detection apparatus according to the first embodiment of the present disclosure.

FIG. 5 is a flowchart illustrating the heart rate detection method according to the first embodiment of the present disclosure.

FIG. 6 is a diagram illustrating a configuration example of a computer in the embodiment of the present disclosure.

### DETAILED DESCRIPTION OF ILLUSTRATIVE EMBODIMENTS

### First Embodiment

A first embodiment of the present disclosure will be described with reference to FIGS. 1 to 5.

FIG. 1 illustrates a part of an ECG waveform measured by using a wearable device. FIG. 1 also illustrates an R-R interval [ms] corresponding to a heart rate at a timing at which an R wave is detected (black rhombus in FIG. 1). Here, for example, the method disclosed in PTL1 can be used to detect the R-R interval.

In this waveform, it is understood that the noise of the ECG waveform is small and the R wave is detected in a substantially appropriate manner in a period of 15:53:30 to 15:53:33, and the noise of the ECG waveform is large and the R wave is not precisely detected in a period of 15:53:20 to 15:53:25.

Next, a method of using an average heart rate (referred to as an “HR” below) as an index for accurate heart rate detection, which is one of methods in the related art, will be described.

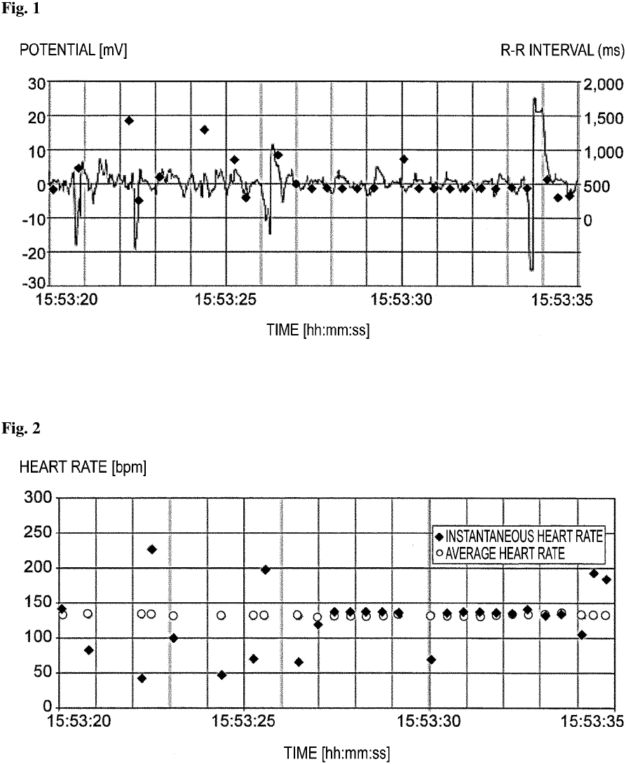

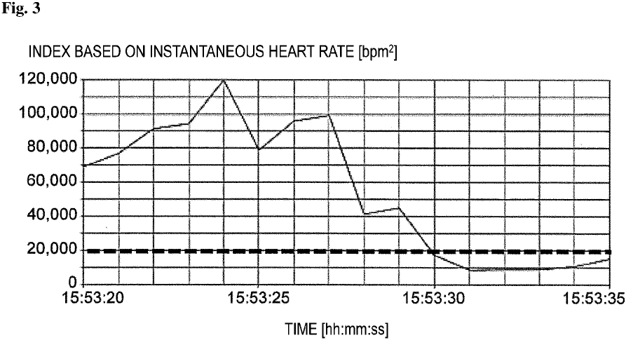

FIG. 2 illustrates an instantaneous heart rate (referred to as an “IHR” below) and an average heart rate HR calculated based on the instantaneous heart rate. In FIG. 2, each black rhombus indicates the instantaneous heart rate, and each white circle indicates the average heart rate.

The instantaneous heart rate IHR is calculated by the equation: the instantaneous heart rate IHR [bpm] = 60,000 ÷ (R-R interval [ms]).

In addition, the average heart rate HR [n] is calculated by the following equation with respect to time-series data IHR [n] of the instantaneous heart rate by using the IIR filter.

$$\text{HR} \left[\text{n}\right] = \left(1 \text{-} \text{a}\right) \times \text{HR} \left[\text{n - 1}\right] + \text{a} \times \text{IHR} \left[\text{n}\right]$$

Here, a is equal to 0.1.

As illustrated in FIG. 2, in the period of 15:53:30 to 15:53:33, the variation in the instantaneous heart rate is small, the average heart rate is also stably observed, and highly reliable data is obtained.

Unfortunately, the instantaneous heart rate varies greatly in the period of 15:53:20 to 15:53:25. The average heart rate having a substantially fixed value is observed. The observed average heart rate is obtained based on the instantaneous heart rate having large variation and thus is unreliable.

This suggests that, even though the average heart rate has a stable value, it may be unreliable. As described above, even though the average heart rate is used as an index for accurate heart rate detection, it is not always possible to accurately detect the heart rate.

Thus, an index based on the instantaneous heart rate is examined focusing on the variation in the instantaneous heart rate. Here, the index based on the instantaneous heart rate is related to a difference of time-series data IHR[n] of the instantaneous heart rate and is calculated by adding the square of (IHR[n] - IHR[n - 1]) of the last 10 beats (heart rate). Here, IHR[n] indicates an instantaneous heart rate at a certain time point t[n]. IHR[n - 1] indicates an instantaneous heart rate at the latest time point t[n - 1] before t[n].

FIG. 3 illustrates a temporal change of the index based on the above-described instantaneous heart rate regarding the waveform illustrated in FIG. 1. The index has a high value in the period of 15:53:20 to 15:53:25, decreases in a period of 15:53:25 to 15:53:30, and has a low value in the period of 15:53:30 to 15:53:35.

In the temporal change of the index, when a reference value is set to 20,000 [bpm²], the index exceeds the reference value in a period of 15:53:20 to 15:53:30 and does not satisfy the criterion. Thus, it is determined that the heart rate measured in the period of 15:53:30 to 15:53:35 is not accurate.

In the period of 15:53:30 to 15:53:35, the index is equal to or less than the reference value, and satisfies the criterion. Thus, it is determined that the heart rate measured in the period of 15:53:30 to 15:53:35 is accurate.

As described above, when the index based on the instantaneous heart rate is used, the index when the ECG waveform noise illustrated in FIG. 1 is large and accurate heart rate detection is not possible, the index has a value more than the reference value. When the ECG waveform noise is small and accurate heart rate detection is possible, the index has a value equal to or less than the reference value.

Thus, use of the index based on the instantaneous heart rate makes it possible to determine whether the measured heart rate is accurate. In other words, it is possible to accurately detect (present) the heart rate.

The present disclosure is based on the knowledge that the heart rate can be accurately detected (presented) by the index based on the instantaneous heart rate.

### Configuration of Heart Rate Detection Apparatus

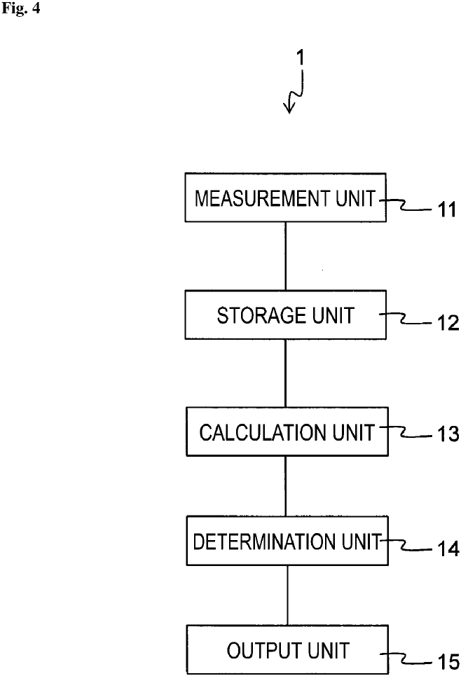

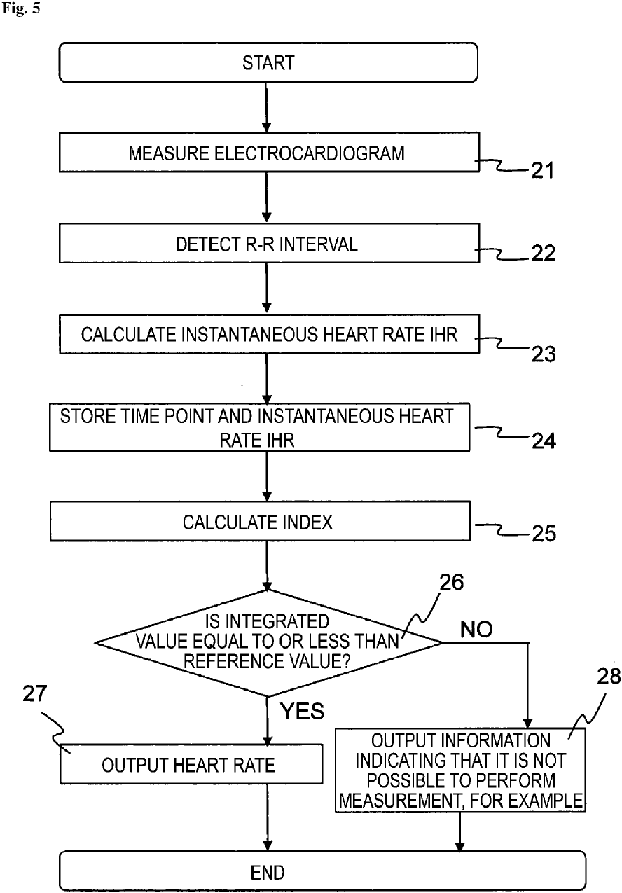

FIG. 4 is a block diagram illustrating an example of a configuration of a heart rate detection apparatus 1 according to the present embodiment. The heart rate detection apparatus 1 includes a measurement unit 11, a storage unit 12, a calculation unit 13, a determination unit 14, and an output unit 15.

The measurement unit 11 measures an ECG waveform, and uses an electrocardiograph in a wearable device as an example.

The storage unit 12 stores an instantaneous heart rate calculated based on an ECG potential measured by the electrocardiograph.

The storage unit 12 stores a time point and the ECG potential measured by the electrocardiograph.

In the calculation unit 13, the time point and the ECG potential measured by the electrocardiograph are acquired from the storage unit 12, and calculation is performed based on the ECG waveform to detect an R-R interval. In addition, the calculation unit 13 calculates the instantaneous heart rate and a numerical value serving as the index.

The determination unit 14 determines (validity) whether the detected heart rate is accurate, by comparing the index to the reference value.

The output unit 15 outputs the calculated heart rate. Information indicating that the heart rate has not been accurately measured, the measured ECG waveform, the time difference value, and the integrated value can also be output.

### Heart Rate Detection Method

FIG. 5 is a flowchart illustrating a heart rate detection method according to the present embodiment.

First, an electrocardiogram (ECG waveform) is measured (Step 21).

Then, an R-R interval is detected by using data of the ECG waveform (Step 22). Here, the R-R interval can be calculated (detected), for example, by being compared to a predetermined threshold value based on a change in a time difference value of the ECG waveform. It is possible to improve detection accuracy, for example, by comparing the heart rate to a predetermined threshold value by using changes in time difference values of a plurality of ECG waveforms (see PTL 1)

Then, the instantaneous heart rate IHR is calculated from the detected R-R interval, by the equation: IHR = 60,000 ÷ (R-R interval [ms]) (Step 23). Here, the instantaneous heart rate is acquired as time-series data IHR[n], from the R-R interval detected at a certain time point t[n].

Then, a time point at which the R-R interval is detected and the instantaneous heart rate IHR calculated from the R-R interval are stored (Step 24).

Then, the index related to the difference of the time-series data IHR[n] of the instantaneous heart rate is calculated by adding the square of (IHR[n] - IHR[n - 1]) of the last 10 beats (heart rate) (Step 25). Here, IHR[n] indicates an instantaneous heart rate at a certain time point t[n]. IHR[n - 1] indicates the instantaneous heart rate at the latest time point t[n - 1] before t[n], and the instantaneous heart rate stored in the storage unit is read and used. As described above, (IHR[n] - IHR[n - 1]) indicates the difference value of the instantaneous heart rate IHR between sampling time points.

Here, an example has been described in which, when the index is calculated, the latest 10 beats (heart rate) are added, but the present disclosure is not limited to this, and a predetermined number of beats may be added.

Then, the calculated index is compared to the reference value, and determination is performed as follows (Step 26).

When the index is equal to or less than the reference value, it is determined that the heart rate calculated from the ECG waveform is accurate.

In this case, the heart rate obtained from the R-R interval detected at the time point t[n] is output (presented) (Step 27).

When the index is more than the reference value, it is determined that the heart rate calculated from the ECG waveform is not accurate.

In this case, the heart rate is not calculated, and information indicating that an accurate heart rate is not measured is output (displayed) (Step 28). Alternatively, the latest accurate heart rate before the time point t[n] may be displayed, or a blank (state where nothing is displayed) may be displayed.

Next, an example of an operation (output) in the heart rate detection method and apparatus according to the present embodiment will be described. Here, the reference value is set to 20,000 [bpm²].

In a case where the ECG waveform illustrated in FIG. 1 is measured, the index illustrated in FIG. 3 is calculated. Then, in the period of time points of 15:53:20 to 15:53:30 in FIG. 3, the reference value is more than 20,000 [bpm²]. Thus, it is determined that the heart rate calculated from the ECG waveform is not accurate. Thus, information indicating that an accurate heart rate is not measured is output (presented). Alternatively, the latest accurate heart rate before the measurement time point is presented. Alternatively, a blank (state in which nothing is displayed) is presented.

In the period of time points of 15:53:30 to 15:53:35 in FIG. 3, the reference value is equal to or less than 20,000 [bpm²]. Thus, it is determined that the heart rate calculated from the ECG waveform is accurate. Thus, the heart rate calculated from the ECG waveform illustrated in FIG. 1 is output (presented).

As described above, according to the present embodiment, it is possible to avoid presenting an abnormal numerical value to the user in a situation where the heart rate is not accurately measured, and to present only an accurate numerical value of the heart rate to the user.

In the present embodiment, the reference value of 20,000 [bpm²] is used, but the present disclosure is not limited to this, and other values may be used.

In the present embodiment, the square of (IHR[n] - IHR[n -1]) is used for the index, focusing on the fact that the difference of the time-series data of the instantaneous heart rate before and after is large, in a case where the reliability of the detected heart rate data is low, but the present disclosure is not limited to this. A difference between the instantaneous heart rate and the average heart rate may be used as the index. In this case, a step of calculating the average heart rate is performed after the instantaneous heart rate is calculated, in the flowchart illustrating the heart rate detection method according to the first embodiment illustrated in FIG. 5.

Alternatively, |IHR[n] - IHR[n - 1]| or an n-th power value of (IHR[n] - IHR[n - 1]) may be used as the index. In addition, an absolute value of the instantaneous heart rate may be used.

As described above, the numerical value based on the instantaneous heart rate can be used as the index.

As described above, according to the present embodiment, it is possible to avoid presenting an abnormal numerical value to the user in a situation where the heart rate is not accurately measured, and to present only an accurate numerical value of the heart rate to the user.

The heart rate detection apparatus according to the present embodiment may be worn on the body of the user as a wearable device.

Alternatively, in the heart rate detection apparatus according to the present embodiment, the measurement unit may be worn on the body of the user as a wearable device, and the storage unit, the calculation unit, and the determination unit may be provided in a smartphone, a server, or the like outside the wearable device. In this case, the heart rate detection apparatus includes a transmission/reception unit in each of the wearable device, an external server, and the like, transmits an ECG waveform measured by the wearable device to the server and the like, and causes the server and the like to perform storing, calculation, determination, and the like. Finally, the heart rate and the like (including information indicating that the heart rate is not measured) may be output to the server and the like, or may be transmitted to the wearable device or the like and output.

In the embodiment according to the present disclosure, the examples have been described in which the latest ECG waveform (ECG potential) is sequentially acquired from the storage unit at a timing of measurement, and the validity of whether the heart rate is accurate is determined. The ECG waveforms may be collectively stored, and then acquired from the storage unit, and the validity of the heart rate may be determined.

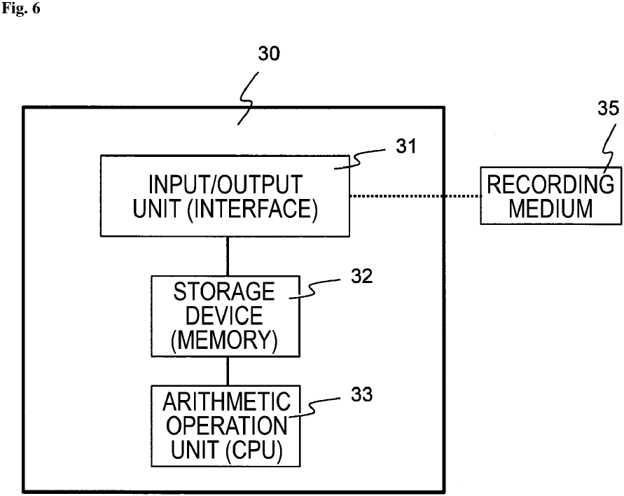

FIG. 6 illustrates a configuration example of a computer 30 in the heart rate detection apparatus according to the embodiment of the present disclosure. The heart rate detection apparatus can be achieved by the computer 30 including a central processing unit (CPU) 33, a storage device (storage unit) 32, and an interface device 31, and programs for controlling the hardware resources. Here, the measurement unit and the output unit in the heart rate detection apparatus according to the embodiment of the present disclosure are connected to the interface device 31. The CPU 33 executes processing in the embodiment of the present disclosure, in accordance with a heart rate detection program stored in the storage device 32. In this manner, the heart rate detection program causes the heart rate detection apparatus to operate.

In the heart rate detection apparatus according to the embodiment of the present disclosure, the computer may be provided inside the device, or at least one of the functions of the computer may be implemented by using an external computer. A storage medium outside the apparatus may also be used as the storage unit, and a heart rate detection program stored in the storage medium may be read and executed. The storage medium includes various magnetic recording media, magneto-optical recording media, CD-ROMs, CD-Rs, and various memories. The heart rate detection program may be supplied to the computer via a communication line such as the Internet.

In the embodiment of the present disclosure, an example of the structure, dimension, material, and the like of each component in the configuration of the heart rate detection apparatus, the heart rate detection method, and the like has been described; however, the present disclosure is not limited thereto. Any structure, dimension, material, and the like may be available as long as the apparatus exhibits the functions and effects of the heart rate detection apparatus and method.

### Industrial Applicability

The present disclosure can be applied to a technique for analyzing a biological signal obtained from an ECG waveform.

### Reference Signs List

1 Heart rate detection apparatus
11 Measurement unit
12 Storage unit
13 Calculation unit
14 Determination unit.

1-8. (canceled)

9. A heart rate detection method comprising:
measuring, by a heart rate detection apparatus, an ECG waveform;
calculating an R-R interval from the ECG waveform;
calculating an instantaneous heart rate from the R-R interval;
calculating an index based on the instantaneous heart rate; and
in response to comparing the index to a reference value, determining whether a heart rate obtained from the R-R interval is to be displayed.

10. The heart rate detection method according to claim 9, wherein the index is based on a difference value of the instantaneous heart rate between sampling time points.

11. The heart rate detection method according to claim 9, further comprising calculating an average heart rate from the instantaneous heart rate, wherein the index is based on a difference value between the instantaneous heart rate and the average heart rate.

12. The heart rate detection method according to claim 9, wherein determining whether the heart rate obtained from the R-R interval is to be displayed comprises:
when the index is equal to or less than the reference value, determining to display the heart rate obtained from the R-R interval; and
when the index is more than the reference value, determining to display a blank.

13. The heart rate detection method according claim 9, wherein determining whether the heart rate obtained from the R-R interval is to be displayed comprises:
when the index is equal to or less than the reference value, determining to display the heart rate obtained from the R-R interval; and
when the index is more than the reference value, determining to display information indicating that an accurate heart rate is not measured.

14. The heart rate detection method according to claim 9, wherein determining whether the heart rate obtained from the R-R interval is to be displayed comprises:
when the index is equal to or less than the reference value, determining to display the heart rate obtained from the R-R interval; and
when the index is more than the reference value, determining to display a latest accurate heart rate before a time point at which the R-R interval is detected.

15. A heart rate detection apparatus comprising:
a measurement circuit configured to measure an ECG waveform;
a calculation circuit configured to:
calculate an R-R interval from the ECG waveform;
calculate an instantaneous heart rate from the R-R interval; and
calculate an index based on the instantaneous heart rate; and
a determination circuit configured to in response to comparing the index to a reference value, determine whether a heart rate obtained from the R-R interval is to be displayed.

16. The heart rate detection apparatus according to claim 15, wherein the index is based on a difference value of the instantaneous heart rate between sampling time points.

17. The heart rate detection apparatus according to claim 15, wherein the calculation circuit is further configured to calculate an average heart rate from the instantaneous heart rate, wherein the index is based on a difference value between the instantaneous heart rate and the average heart rate.

18. The heart rate detection apparatus according to claim 15, wherein the determination circuit is configured to:
when the index is equal to or less than the reference value, determine to display the heart rate obtained from the R-R interval; and
when the index is more than the reference value, determine to display a blank.

19. The heart rate detection apparatus according to claim 15, wherein the determination circuit is configured to:
when the index is equal to or less than the reference value, determining to display the heart rate obtained from the R-R interval; and
when the index is more than the reference value, determining to display information indicating that an accurate heart rate is not measured.

20. The heart rate detection apparatus according to claim 15, wherein the determination circuit is configured to:
when the index is equal to or less than the reference value, determining to display the heart rate obtained from the R-R interval; and
when the index is more than the reference value, determining to display a latest accurate heart rate before a time point at which the R-R interval is detected.

21. The heart rate detection apparatus according to claim 15, wherein the determination circuit is configured to:
when the index is equal to or less than the reference value, determine to display the heart rate obtained from the R-R interval; and
when the index is more than the reference value, determine to display a latest accurate heart rate before a time point at which the R-R interval is detected.

22. A non-transitory memory storing a heart rate detection program causing a heart rate detection apparatus to operate, the heart rate detection program causing the heart rate detection apparatus to perform:
measuring an ECG waveform;
calculating an R-R interval from the ECG waveform;
calculating an instantaneous heart rate from the R-R interval;
calculating an index based on the instantaneous heart rate; and
in response to comparing the index to a reference value, determining whether a heart rate obtained from the R-R interval is to be displayed.

23. The non-transitory memory storing the heart rate detection program according to claim 22, wherein the index is based on a difference value of the instantaneous heart rate between sampling time points.

24. The non-transitory memory storing the heart rate detection program according to claim 22, wherein the heart rate detection program further causes the heart rate detection apparatus to perform:
calculating an average heart rate from the instantaneous heart rate, wherein the index is based on a difference value between the instantaneous heart rate and the average heart rate.

25. The non-transitory memory storing the heart rate detection program according to claim 22, wherein the heart rate detection program further causes the heart rate detection apparatus to perform:
when the index is equal to or less than the reference value, determining to display the heart rate obtained from the R-R interval; and
when the index is more than the reference value, determining to display a blank.

26. The non-transitory memory storing the heart rate detection program according to claim 22, wherein the heart rate detection program further causes the heart rate detection apparatus to perform:
when the index is equal to or less than the reference value, determining to display the heart rate obtained from the R-R interval; and
when the index is more than the reference value, determining to display information indicating that an accurate heart rate is not measured.

27. The non-transitory memory storing the heart rate detection program according to claim 22, wherein the heart rate detection program further causes the heart rate detection apparatus to perform:
when the index is equal to or less than the reference value, determining to display the heart rate obtained from the R-R interval; and
when the index is more than the reference value, determining to display a latest accurate heart rate before a time point at which the R-R interval is detected.
